(19) United States
(12) Patent Application Publication
(10) Pub. No.: US 2018/0289752 A1
(43) Pub. Date: Oct. 11, 2018

(54) Spore-Based Probiotic Composition for Reduction of Dietary Endotoxemia and Related Methods

(71) Applicant: Physician's Exclusive, LLC, Glenview, IL (US)

(72) Inventors: Kiran Krishnan, Glenview, IL (US); Dale Kriz, Phoenix, AZ (US); Thomas Bayne, Glenview, IL (US)

(21) Appl. No.: 15/947,521

(22) Filed: Apr. 6, 2018

Related U.S. Application Data
(60) Provisional application No. 62/482,657, filed on Apr. 6, 2017.

(51) Int. Cl. A61K 35/742; A23L 31/10; A23L 33/135; A23L 33/14; A61K 35/747; A61P 31/04

(52) U.S. Cl. CPC A61K 35/742; A23L 31/10; A23L 33/135; A23L 33/14; A23V 2200/328; A61P 31/04; A23V 2002/00; A23V 2200/3204; A61K 35/747

(57) ABSTRACT

A method of treating metabolic endotoxemia comprising identifying a subject having post-prandial dietary endotoxemia and administering an effective amount of a spore-based probiotic. While any spore-based probiotic may be used, the probiotic supplement may comprise Bacillus indicus (HU36), Bacillus subtilis (HU58), Bacillus coagulans, Bacillus licheniformis, and Bacillus clausii. One or more of a level of blood endotoxin, triglyceride, post-prandial insulin, post-prandial ghrelin level, MCP-1, GM-CSF, IL-12p70, IL-13, IL-1beta, IL-4, IL-5, IL-6, IL-7, IL-8, and TNF-α is observed as being lower after spore-based probiotic supplementation when compared to placebo. At least one of post-prandial leptin and IL-10 is observed as being higher after spore-based probiotic supplementation when compared to placebo.

### CROSS REFERENCE TO RELATED APPLICATIONS

This application claims priority to U.S. Application Ser. No. 62/482,657 entitled “Spore-Based Probiotic Composition for Reduction of Dietary Endotoxemia and Related Methods” to Kiran Krishnan, et al, filed on Apr. 6, 2017, the contents of which are herein incorporated by reference in its entirety.

### TECHNICAL FIELD

The present disclosure relates to the field of spore-based probiotic compositions. A spore-based probiotic composition is provided that comprises at least one viable probiotic microorganism having a biological or therapeutic activity in the gastrointestinal tract. Also provided are methods of producing spore-based probiotic compositions.

### BACKGROUND

Probiotic microorganisms are live microbial preparations that may be administered to a subject in order to confer a beneficial effect, such as restoring or improving the composition of intestinal microflora. Probiotics are typically provided as dietary supplements containing potentially beneficial bacteria or yeast and are widely consumed in foods as well as in capsules and powders. Generally, lactic acid bacteria including Lactobacillus and Bifidobacterium are used as probiotics but other genus are also used including Lactococcus, Propionibacterium, Bacillus, Saccharomyces as well as strains of Escherichia. Within these genus, many species and strains have been reported to have probiotic properties. The most common vehicles for the delivery of probiotics are dairy products and probiotic fortified foods. However, powders, tablets and capsules containing probiotics are also available.

Incidence of gastrointestinal (GI) distress and permeability has increased in prominence in modern society due in large part to the excessive consumption of highly processed, calorie dense, commercially available foods. These same dietary choices coupled with low physical activity are believed to be the primary causes underlying the current obesity epidemic. Recent efforts have focused on the use of over-the-counter probiotics (typically Lactobacillus and Bifidobacterium) to address symptoms associated with GI abnormalities. Existing probiotic supplementation does not yield consistent results and may only be effective for individuals having a pre-existing GI abnormality. Further complicating oral probiotic supplementation efforts is the fact that few traditional probiotic supplements (i.e. Lactobacillus and Bifidobacterium) delivery fully viable bacteria to the small intestine.

Dietary or metabolic endotoxemia is a condition that affects approximately one third of individuals living in Western society. It is characterized by increased serum endotoxin concentration during the first five hours of the post-prandial period following consumption of a meal with a high-fat, high-calorie content. Long-term repeated dietary endotoxemia may increase the risk of developing a variety of chronic diseases via an inflammatory etiology.

### SUMMARY

In some implementations, a method of treating metabolic endotoxemia may comprise identifying a subject having metabolic endotoxemia and administering an effective amount of a spore-based probiotic. The spore-based probiotic may comprise spores selected from the group of genus consisting of: Lactobacillus, Bifidobacterium, Lactococcus, Propionibacterium, Bacillus, Enterococcus, Escherichia, Streptococcus, Pediococcus, Saccharomyce. The spore-based probiotic may comprise spores selected from the group consisting of Bacillus indicus (HU36), Bacillus subtilis (HU58), Bacillus coagulans, Bacillus licheniformis, and Bacillus clausii. The method of may further comprise reducing at least one of a post-prandial insulin level, a post-prandial ghrelin level, and an MCP-1 level. The method may further comprise reducing a level of at least one of GM-CSF, IL-12p70, IL-13, IL-1beta, IL-4, IL-5, IL-6, IL-7, IL-8, and TNF-α. The method may further comprise increasing at least one of a post-prandial leptin level and an IL-10 level. The spore-based probiotic may comprise spores having a survival rate between about 75% and 99% after exposure to gastric acid. The spore-based probiotic may comprise spores having a survival rate greater than about 90% after exposure to gastric acid. The spore-based probiotic may be in at least one of a liquid form, a pill form and a food product form. The subject may experiences at least one of a reduction in triglyceride and a post-prandial reduction of an endotoxin after administration of the effective amount of the spore-based probiotic. The endotoxin may comprise lipopolysaccharide (LPS).

Implementations of a method of reducing a blood endotoxin level may comprise identifying a subject having an increased post-prandial level of a blood endotoxin and administering an effective amount of a spore-based probiotic. The spore-based probiotic may comprise spores selected from the group of genus consisting of Lactobacillus, Bifidobacterium, Lactococcus, Propionibacterium, Bacillus, Enterococcus, Escherichia, Streptococcus, Pediococcus, Saccharomyce. The spore-based probiotic may comprise spores selected from the group consisting of Bacillus indicus (HU36), Bacillus subtilis (HU58), Bacillus coagulans, Bacillus licheniformis, and Bacillus clausii. The spore-based probiotic may comprise spores having a survival rate between about 75% and 99% after exposure to gastric acid. The spore-based probiotic may comprise spores having a survival rate greater than about 90% after exposure to gastric acid. The method may further comprise reducing at least one of an endotoxin level and a triglyceride level of the subject after administration of the effective amount of the spore-based probiotic. The endotoxin may comprise lipopolysaccharide (LPS). The method may further comprise reducing at least one of a post-prandial insulin level, a post-prandial ghrelin level, and an MCP-1 level after administration of the effective amount of the spore-based probiotic. The method may further comprise reducing a level of at least one of GM-CSF, IL-12p70, IL-13, IL-1beta, IL-4, IL-5, IL-6, IL-7, IL-8, and TNF-α after administration of the effective amount of the spore-based probiotic. The method may further comprise increasing at least one of a post-prandial leptin level and an IL-10 level after administration of the effective amount of the spore-based probiotic. The spore-based probiotic may be in at least one of a liquid form, a pill form, and a food product form.

### BRIEF DESCRIPTION OF THE DRAWINGS

FIG. 1 provides a consort diagram indicating a number of participants matriculated through the study of Example 1.

FIGS. 2A-B depict serum endotoxin and triglyceride response to consumption of a high-fat, high-calorie meal in accordance with the study of Example 1.

FIGS. 3A-C depict serum IL-12p70, IL-1β, and post-prandial ghrelin response to consumption of a high-fat, high-calorie meal in accordance with the study of Example 1.

FIGS. 4A-C depict serum IL-6, IL-8, and MCP-1 response to consumption of a high-fat, high-calorie meal in accordance with the study of Example 1.

FIGS. 5A-I depict serum GM-CSF, IL-10, IL-13, IL-4, IL-5, IL-7, TNF-α, post-prandial insulin, and leptin response to consumption of a high-fat, high-calorie meal in accordance with the study of Example 1.

### DETAILED DESCRIPTION

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

As used herein, an “effective amount” or an “amount effective for” is defined as an amount effective, at dosages and for periods of time necessary, to achieve a desired biological result, such as reducing, preventing, or treating a disease or condition and/or inducing a particular beneficial effect. The effective amount of compositions of the disclosure may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present disclosure may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

As used herein, the term “subject” or “patient” refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some implementations, the subject may be a mammal. In other implementations, the subject may be a human.

The present disclosure provides probiotic compositions, methods of producing these probiotic compositions, and methods of treating various indications by administering an effective about of the probiotic compositions to a subject in need thereof. More specifically, a composition of two or more probiotic strains creates an unexpected synergy that reduces or eliminates post-prandial endotoxemia, lowers triglycerides, and may alleviate glucose intolerance as discussed in detail in the remainder of this disclosure. These effects have been experimentally verified based on supplementation of study participants with a composition comprising two or more colonizing probiotic bacterial strains which may be spore-based probiotic bacterial strains.

The probiotic compositions may contain a probiotic microorganism which in some applications may be a spore-based probiotic organism selected from the following genus: Lactobacillus, Bifidobacterium, Lactococcus, Propionibacterium, Bacillus, Enterococcus, Escherichia, Streptococcus, Pediococcus, Saccharomyce. In certain aspects, the probiotic microorganism is at least one of Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium sp., Bifidobacterium longum, Bifidobacterium infantis, Bifidobacterium animalis, Bifidobacterium bifidum, Bifidobacterium adelocentis, Bifidobacterium lactis, Bacillus subtilis, Bacillus coagulans, Bacillus licheniformis, Enterococcus faecalis, Enterococcus faecium, Lactococcus lactis, Streptococcus salivarius, Saccharomyces cerevisiae, and Saccharomyces boulardii. The probiotic microorganism may be in the form of spores or in a vegetative state.

In some implementations, the concentration of the probiotic microorganism in the composition may be at least about 1×10⁹ cfu/g, at least about 2×10⁹ cfu/g, at least about 3×10⁹ cfu/g, at least about 4×10⁹ cfu/g, at least about 5×10⁹ cfu/g, at least about 6×10⁹ cfu/g, at least about 7×10⁹ cfu/g, at least about 8×10⁹ cfu/g, at least about 9×10⁹ cfu/g, at least about 1×10¹⁰ cfu/g, at least about 2×10¹⁰ cfu/g, at least about 3×10¹⁰ cfu/g, at least about 4×10¹⁰ cfu/g, at least about 5×10¹⁰ cfu/g, at least about 6×10¹⁰ cfu/g, at least about 7×10¹⁰ cfu/g, at least about 8×10¹⁰ cfu/g, at least about 9×10¹⁰ cfu/g, or at least about 1×10¹¹ cfu/g.

The spore-based probiotic supplement may comprise spores having a survival rate within any of the following ranges after exposure to gastric acid: about 75% to about 99%, about 80% to about 95%, about 85% to about 90%, and greater than about 90%.

The spore-based probiotic supplement may comprise a number of spores within any of the following ranges: about 1 billion to about 10 billion spores, about 1.5 billion spores to about 9.5 billion spores, about 2 billion spores to about 9 billion spores, about 2.5 billion spores to about 8 billion spores, about 3 billion spores to about 7 billion spores, about 3.5 billion spores to about 6 billion spores, about 3.5 billion spores to about 6 billion spores, about 3.5 billion spores to about 5 billion spores and about 3.5 billion spores to about 4.5 billion spores.

The spore-based probiotic supplement may comprise a liquid or pill form or may be added to a food product. In one implementation, about 1×10¹⁰ cfu of microorganism is present in each gram of bulk, dried raw powder where each gram contains about 60% or less of bacterial mass and about 40% carrier system. In other implementations, each gram contains about 70% or less of bacterial mass and about 30% carrier system, about 80% or less of bacterial mass and about 20% carrier system, about 90% or less of bacterial mass and about 10% carrier system, about 50% or less of bacterial mass and about 50% carrier system, about 40% or less of bacterial mass and about 60% carrier system, about 30% or less of bacterial mass and about 70% carrier system, about 20% or less of bacterial mass and about 80% carrier system, or about 10% or less of bacterial mass and about 90% carrier system.

It is commonly believed that the onset and progression of chronic disease results from the accumulation of transient changes in ones' health that result from lifestyle choices. Unfortunately, the current literature has yet to define the quantity of transient change that must be accumulated to cause disease onset. Instead, previous studies have attempted to use lifestyle modifications (i.e. nutrition, physical activity, etc.) to minimize negative changes in health. One such problem, especially in western cultures, is the wide accessibility to high-fat, high-calorie meals, creating an environment where excessive, low-quality nutritional habits are the norm. In these diets, elevated post-prandial endotoxin and triglyceride are consistently reported as problematic changes.

Dietary or metabolic endotoxemia occurs when one's dietary consumption causes disruption in either GI permeability, the microbiota profile, or both. Dietary endotoxemia transiently increases systemic inflammation, which chronically may increase one's risk of a variety of diseases. It is known that consumption of a single, high-fat, high-calorie meal is associated with an increase in serum endotoxin, triglycerides, metabolic biomarkers, inflammatory cytokines, endothelial microparticles, and monocyte adhesion molecules. The post-prandial time course varies for each biomarker, but generally the transient changes occur during the first five hours of the post-prandial period. For the purposes of this disclosure, it is to be understood that the terms “dietary endotoxemia,” “metabolic endotoxemia,” and “post-prandial endotoxemia” are used interchangeably.

It is also known that the consumption of a high-fat meal causes transient biological changes that are consistent with a transient increase in risk of atherosclerosis. These changes combined with a post-prandial increase in serum triglycerides creates a milieu that favors foam cell formation and the development of atherosclerotic plaques.

Implementations of the methods and compositions disclosed herein may comprise a spore-based probiotic. A spore-based probiotic is comprised of endosomes which are highly resistant to acidic pH, are stable at room temperature, and deliver a much greater quantity of high viability bacteria to the small intestine that traditional probiotic supplements. Traditional micro-encapsulation uses live microorganisms which are then micro-encapsulated in an effort to protect the microorganisms; however, this is a process that inherently leads to the eventual death of the microorganisms thereby reducing the efficacy of the microorganisms. Using spore-based microorganisms that have been naturally microencapsulated to form endosomes may be preferable as these microorganisms are dormant and do not experience a degradation in efficacy over time. These spore-based microorganisms are also particularly thermal stable and can survive UV pasteurization so they are also able to be added to food products or beverages prior to thermal exposure or UV pasteurization without experiencing a degradation in efficacy over time.

### Micro-Encapsulation

In certain implementations, the probiotic microorganisms are microencapsulated prior to addition to the probiotic compositions. Micro-encapsulation is a process in which tiny particles or droplets are surrounded by a coating to give small capsules of many useful properties. In a relatively simple form, a microcapsule is a small sphere with a uniform wall around it. The material inside the microcapsule is referred to as the core, internal phase, or fill, whereas the wall is sometimes called a shell, coating, or membrane. Most microcapsules have diameters between a few micrometers and a few millimeters.

The definition has been expanded, and includes most foods. Every class of food ingredient has been encapsulated; flavors are the most common. The technique of microencapsulation depends on the physical and chemical properties of the material to be encapsulated. See Jackson L. S.; Lee K. (Jan. 1, 1991). “Microencapsulation and the food industry”. Lebensmittel-Wissenschaft Technologie.

Many microcapsules however bear little resemblance to these simple spheres. The core may be a crystal, a jagged adsorbent particle, an emulsion, a Pickering emulsion, a suspension of solids, or a suspension of smaller microcapsules. The microcapsule even may have multiple walls.

Various techniques may be used to produce microcapsules. These include pan coating, air-suspension coating, centrifugal extrusion, vibrational nozzle, spray-drying, ionotropic gelation, interfacial polycondensation, interfacial cross-linking, in situ polymerization, and matrix polymerization as described below.

### Pan Coating

The pan coating process, widely used in the pharmaceutical industry, is among the oldest industrial procedures for forming small, coated particles or tablets. The particles are tumbled in a pan or other device while the coating material is applied slowly.

### Air-Suspension Coating

Air-suspension coating, first described by Professor Dale Eavin. Wurster at the University of Wisconsin in 1959, gives improved control and flexibility compared to pan coating. In this process the particulate core material, which is solid, is dispersed into the supporting air stream and these suspended particles are coated with polymers in a volatile solvent leaving a very thin layer of polymer on them. This process is repeated several hundred times until the required parameters such as coating thickness, etc., are achieved. The air stream which supports the particles also helps to dry them, and the rate of drying is directly proportional to the temperature of the air stream which can be modified to further affect the properties of the coating.

The re-circulation of the particles in the coating zone portion is effected by the design of the chamber and its operating parameters. The coating chamber is arranged such that the particles pass upwards through the coating zone, then disperse into slower moving air and sink back to the base of the coating chamber, making repeated passes through the coating zone until the desired thickness of coating is achieved.

### Centrifugal Extrusion

Liquids are encapsulated using a rotating extrusion head containing concentric nozzles. In this process, a jet of core liquid is surrounded by a sheath of wall solution or melt. As the jet moves through the air it breaks, owing to Rayleigh instability, into droplets of core, each coated with the wall solution. While the droplets are in flight, a molten wall may be hardened or a solvent may be evaporated from the wall solution. Since most of the droplets are within +10% of the mean diameter, they land in a narrow ring around the spray nozzle. Hence, if needed, the capsules can be hardened after formation by catching them in a ring-shaped hardening bath. This process is excellent for forming particles 400-2,000 μm in diameter. Since the drops are formed by the breakup of a liquid jet, the process is only suitable for liquid or slurry. A high production rate can be achieved, i.e., up to 22.5 kg (50 lb) of microcapsules can be produced per nozzle per hour per head. Heads containing 16 nozzles are available.

### Vibrational Nozzle

Core-Shell encapsulation or Microgranulation (matrix-encapsulation) can be done using a laminar flow through a nozzle and an additional vibration of the nozzle or the liquid. The vibration has to be done in resonance of the Rayleigh instability and leads to very uniform droplets. The liquid can consist of any liquids with limited viscosities (0-10,000 mPa·s have been shown to work), e.g. solutions, emulsions, suspensions, melts etc. The soldification can be done according to the used gelation system with an internal gelation (e.g. sol-gel processing, melt) or an external (additional binder system, e.g. in a slurry). The process works very well for generating droplets between 20-10,000 μm, applications for smaller and larger droplets are known. The units are deployed in industries and research mostly with capacities of 1-20,000 kg per hour (2-44,000 lb/h) at working temperatures of 20-1500° C. (68-2732° F.) (room temperature up to molten silicon). Nozzles heads are available from one up to several hundred thousand are available.

### Spray-Drying

Spray drying serves as a microencapsulation technique when an active material is dissolved or suspended in a melt or polymer solution and becomes trapped in the dried particle. The main advantages are the ability to handle labile materials because of the short contact time in the dryer, in addition, the operation is economical. In modern spray dryers the viscosity of the solutions to be sprayed can be as high as 300 mPa·s. Applying This technique along with the use of supercritical Carbon Dioxide, also sensitive materials like proteins can be encapsulated.

### Ionotropic Gelation

The coacervation-phase separation process consists of three steps carried out under continuous agitation:
1. Formation of 3 immiscible chemical phases: liquid manufacturing vehicle phase, core material phase and coating material phase.
2. Deposition of coating: core material is dispersed in the coating polymer solution. Coating polymer material coated around core. Deposition of liquid polymer coating around core by polymer adsorbed at the interface formed between core material and vehicle phase.
3. Rigidization of coating: coating material is immisible in vehicle phase and it gets rigid form. It done by thermal, cross-linking, or dissolvation techniques.

### Interfacial Polycondensation

In Interfacial polycondensation, the two reactants in a polycondensation meet at an interface and react rapidly. The basis of this method is the classical Schotten-Baumann reaction between an acid chloride and a compound containing an active hydrogen atom, such as an amine or alcohol, polyesters, polyurea, polyurethane. Under the right conditions, thin flexible walls form rapidly at the interface. A solution of the pesticide and a diacid chloride are emulsified in water and an aqueous solution containing an amine and a polyfunctional isocyanate is added. Base is present to neutralize the acid formed during the reaction. Condensed polymer walls form instantaneously at the interface of the emulsion droplets.

### Interfacial Cross-Linking

Interfacial cross-linking is derived from interfacial polycondensation, and was developed to avoid the use of toxic diamines, for pharmaceutical or cosmetic applications. In this method, the small bifunctional monomer containing active hydrogen atoms is replaced by a biosourced polymer, like a protein. When the reaction is performed at the interface of an emulsion, the acid chloride reacts with the various functional groups of the protein, leading to the formation of a membrane. The method is very versatile, and the properties of the microcapsules (size, porosity, degradability, mechanical resistance). Flow of artificial microcapsules in microfluidic channels:

### In-Situ Polymerization

In a few microencapsulation processes, the direct polymerization of a single monomer is carried out on the particle surface. In one process, e.g. Cellulose fibers are encapsulated in polyethylene while immersed in dry toluene. Usual deposition rates are about 0.5 μm/min. Coating thickness ranges 0.2-75 μm (0.0079-3.0 mils). The coating is uniform, even over sharp projections. Protein microcapsules are biocompatible and biodegradable, and the presence of the protein backbone renders the membrane more resistant and elastic than those obtained by interfacial polycondensation.

### Matrix Polymerization

In a number of processes, a core material is imbedded in a polymeric matrix during formation of the particles. A simple method of this type is spray-drying, in which the particle is formed by evaporation of the solvent from the matrix material. However, the solidification of the matrix also can be caused by a chemical change.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

### EXAMPLES

### Example 1. Reduction of Triglycerides, Gherlin, and Inflammatory Markers Using Spore-Based Probiotic Supplementation

Determination of Sample Size:

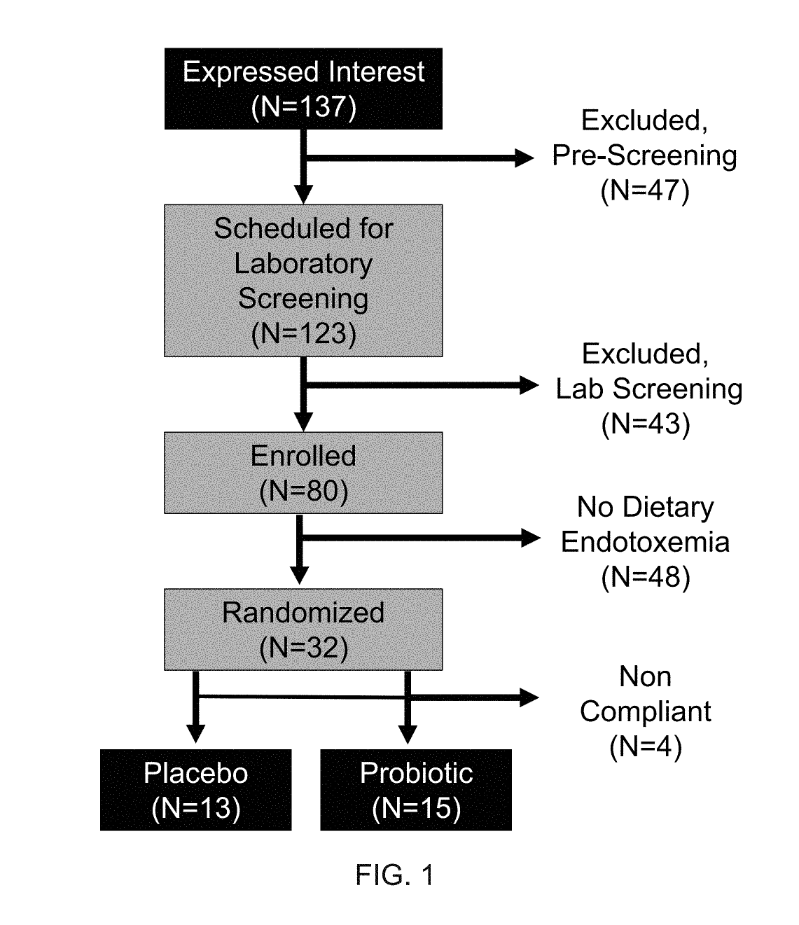

FIG. 1 is a consort diagram that presents the progression of subjects through the intervention of Example 1. A screening protocol was developed to identify individuals who presented with post-prandial endotoxemia at baseline, which may be a hallmark sign of intestinal permeability and “leaky gut” syndrome. It was identified that only 2 of 6 subjects (“responders”) had a measurable dietary endotoxemia response (i.e. at least a 5-fold increase from pre-meal values at 5 hour post-prandial). “Responder” subjects experienced a 30% reduction in serum endotoxin, typically lipopolysaccharide (LPS), at 5 hour post-prandial following a 30 day probiotic intervention. Statistically this reduction resulted in a moderate effect size (0.40). In comparison, the “non-responder” subjects (i.e. <1-fold increase from pre-meal values at 5 hour post-prandial) had <2% decrease in serum endotoxin at 5 hour post-prandial following 30 day of spore-based probiotic intervention. It should be noted that “non-responders” likely have a protective microbiome, while “responders” likely have a non-protective microbiome, which makes them good candidates for treatment with a spore-based probiotic. Based on these criteria, a minimum of N=10 “responders” were enrolled in placebo and spore-based probiotic groups (N=20 total) in order to achieve at least 80% statistical power to detect an associated probiotic effect. Initially, 65 individuals were enrolled in a study for post-prandial dietary endotoxemia screening. As the study progressed, the prevalence of the “responder” phenotype was much smaller (<33%) than observed in a prior, proof-of-concept study. As such, 80 subjects were screened in order to identify 25 that had the “responder” phenotype (31% prevalence) as shown below in Table 1. The individuals with the “responder” phenotype were then randomized to participate in either the spore-based probiotic supplementation group or the placebo group.

TABLE 1
Subject Characteristics

| | Placebo | Probiotic |
|---|---|---|
| Characteristic | (N = 13) | (N = 15) |
| Age (y) | 21.8 ± 0.7 | 21.2 ± 0.5 |
| Height (cm) | 167.9 ± 3.2 | 170.8 ± 2.7 |
| Body Mass (kg) | 74.2 ± 6.6 | 71.2 ± 3.1 |
| Body Mass Index (kg/cm²) | 25.9 ± 1.5 | 24.3 ± 0.9 |
| Body Fat (%) | 27.8 ± 4.1 | 25.2 ± 3.0 |
| Fat Mass (kg) | 21.0 ± 4.3 | 17.3 ± 2.4 |
| Lean Mass (kg) | 50.1 ± 3.8 | 50.0 ± 3.7 |
| Bone Mineral Mass (kg) | 2.9 ± 0.2 | 2.9 ± .1 |
| Resting Energy Expenditure (kcal/d) | 2243 ± 304 | 2071 ± 108 |

Values represent group mean ± standard error of the mean (SEM). No significant differences existed between groups with respect to subject characteristics.

Additional Subject Screening:

Prior to testing for the post-prandial endotoxemia response, subjects also completed a series of other tests to exclude for other pre-existing conditions. Screening included measurement of body composition (DEXA scan), medical history assessment, and resting metabolic rate (RMR, indirect calorimetry). Oxygen consumption (VO2) during the RMR assessment was calculated using automated analysis of expired respiratory air using a metabolic cart (MGC Diagnostics Ultima; St. Paul, Minn.). Body composition was determined using a whole body DEXA scan, followed by analysis using GE whole body software (Lunar Prodigy; USA). Subjects who were currently taking or had taken in the previous 6-months medications for the treatment of metabolic disease, antibiotics, probiotic supplements, anti-inflammatory medications, and/or daily consumed at least three serving of yogurt were excluded from further participation. Within the medical history, subjects who were currently being treated for metabolic disease (i.e. diabetes mellitus), currently being treated for cardiovascular disease, and/or were obese (by BMI and/or percent body fat from DEXA) were also excluded. Individuals who met the initial screening criteria were scheduled to consume the experimental meal challenge on a separate day. The experimental meal challenge was used to identify subjects with a dietary endotoxin response that were considered “responders.” Individuals classified as “responders” were enrolled in the supplementation phase of the study.

Identification of “Responders” Experimental Meal Challenge:

Subjects reported to the laboratory between 0600 and 1000 following an overnight fast (>8-h) and abstention from exercise (>24-h). Following collection of a pre-meal blood sample, subjects were provided a high-fat meal (85% of the daily fat RDA and 65% of the daily calorie needs) that was adjusted based on the participant's measured daily caloric needs (based on measured RMR). Thin crust cheese pizza from a local vendor was used as the high-fat meal source. The meal composition is summarized in Table 2, below:

TABLE 2
Meal Composition

| | Placebo | Probiotic |
|---|---|---|
| Component | (N = 13) | (N = 15) |
| Total Calories | 1630.4 ± 134.4 | 1644.7 ± 94.5 |
| (kcal) | | |
| Total Caloric Needs | 72% | 79% |
| (% of RMR) | | |
| Servings (#) | 6.3 ± 0.5 | 6.4 ± 0.4 |
| Fat (g) | 88.8 ± 7.3 | 89.6 ± 5.1 |
| Fat (kcal) | 799.3 ± 6.6 | 806.4 ± 46.3 |
| Saturated Fat (g) | 31.7 ± 2.6 | 32.0 ± 1.8 |
| Trans Fat (g) | 0.0 | 0.0 |
| Protein (g) | 69.8 ± 5.8 | 70.4 ± 4.0 |
| Carbohydrate (g) | 145.9 ± 12.0 | 147.2 ± 8.5 |
| Carbohydrate (kcal) | 583.6 ± 48.1 | 588.8 ± 33.8 |
| Cholesterol (mg) | 152.3 ± 12.5 | 153.6 ± 8.8 |
| Sodium (mg) | 2911.9 ± 240.0 | 2937.4 ± 168.8 |

Values represent group mean ± standard error of the mean (SEM). No significant differences existed between groups with respect to meal composition

Blood samples were measured for endotoxin concentration after the meal and only those subjects whose endotoxin level increased by >5-fold at 5 hour post-prandial were classified as “responders” and enrolled in the supplementation phase of the study. This same experimental meal challenge was completed at the end of the supplementation period to assess the effectiveness of spore-based probiotic supplementation at modifying the serum endotoxin response. Individuals who have the “responder” phenotype (i.e. GI permeability), will elicit an endotoxin response to any type of meal; however, the response is even more pronounced with a high-fat, high-calorie meal, which is why this type of meal was selected for this study.

Supplementation Conditions:

“Responder” subjects were randomized to either a placebo (rice flour) or spore-based probiotic condition. The spore-based probiotic used in the present study was commercially manufactured and included 4 billion spores from the following gram-positive, spore-forming strains: Bacillus indicus (HU36), Bacillus subtilis (HU58), Bacillus coagulans, and Bacillus licheniformis, Bacillus clausii. Subjects were instructed to consume 2 capsules each day for a total of 30 day. Subjects were asked to promptly report any missed doses. Based on subject reporting, efficacy of intake was >95% for the study period. All group assignments were completed using double-blind procedures. Subjects were instructed to maintain their habitual dietary and lifestyle habits during the study. Subjects were asked to promptly report deviations from their habitual habits as these may have resulted in external error in our experimental model.

Blood Sample Collection:

Venous blood samples were collected prior to the high-fat meal (PRE), 3 hour, and 5 hour post meal from a peripheral arm vein into an evacuated serum tube. Serum tubes were held at room temperature for 30-min to allow for clotting. Serum was separated by centrifugation and frozen at −80° C. until additional analysis.

Dietary Endotoxin Measurement:

Serum was analyzed for endotoxin concentration using a commercially available kinetic limulus amebocyte lysate (LAL) assay. Briefly, serum samples were diluted 1:100 in endotoxin-free water and heated at 70° C. for 15-min to remove contaminating proteases. Treated samples were then analyzed in triplicate using an automated chemistry analyzer to determine endotoxin concentration against an e. coli endotoxin standard.

Serum Triglyceride Measurement:

Serum was analyzed in triplicate for triglyceride concentration using an endpoint enzymatic assay on an automated chemistry analyzer.

Exploratory Disease Risk Biomarkers:

Previously frozen serum samples were analyzed as previously described. Briefly, post-prandial ghrelin, post-prandial insulin, leptin, MCP-1, GM-CSF, IL-4, IL-5, IL-6, IL-7, IL-8, IL-10, IL-12(p70), IL-13, and TNF-α were measured in duplicate using a commercially available bead-based multiplex and an automated analyzer. TNF stands for tumor necrosis factor and IL stands for interleukin. Raw data files were used to calculate unknowns from standards using Milliplex Analyst software (MilliporeSigma).

Statistical Analysis and Data Visualization:

Prior to formal statistical testing, data was assessed for normality. Non-normal data was log-transformed to stabilize this assumption prior to formal testing. Data was analyzed using a condition (placebo or probiotic)×experiment time (baseline and 30 day post)×meal time (pre, 3 hour, and 5 hour post) analysis of variance (ANOVA) with repeated measurements on the 2nd and 3rd factors. P-values were adjusted using the Huygh-Feldt method to account for the repeated measures design. Significance was set at P<0.05. Location of significant effects was determined using separate t-tests with a Bonferroni correction for multiple comparisons.

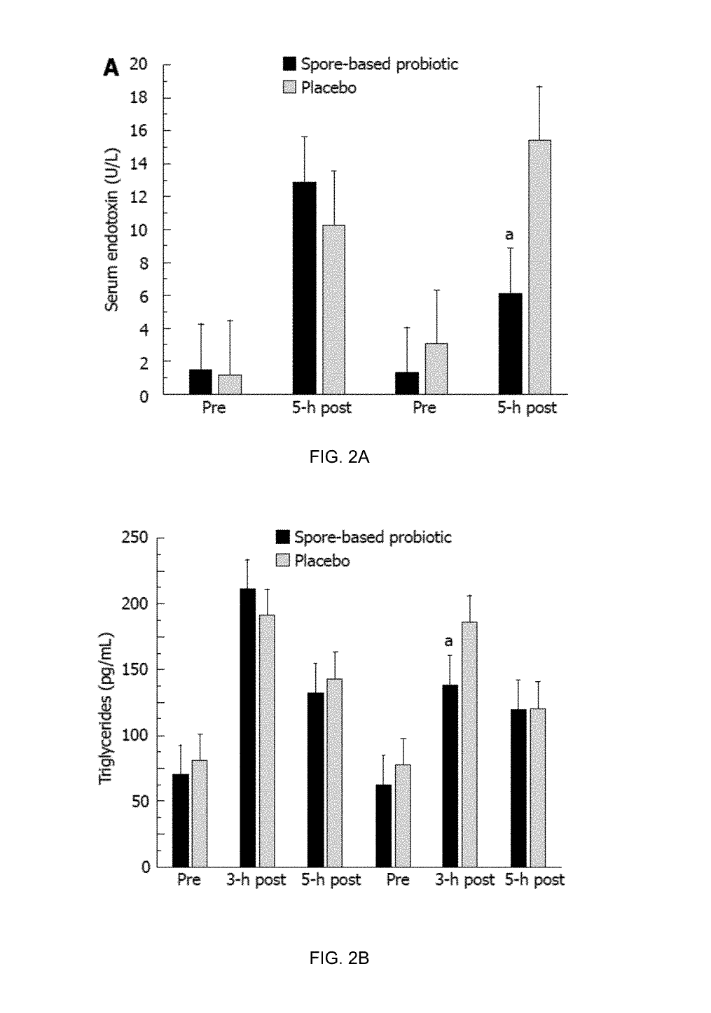

In order to visualize the responses collectively, all of the responses were log transformed to normalize the various biomarkers to a similar scale. Three radar plots (one for each sampling time point) were then created. Each plot contains the log transformed variable response at baseline and 30 days post-supplementation and a third line for the fold-change from pre-meal response as shown in FIGS. 2A-B.

FIGS. 2A-B show serum endotoxin (A) and triglyceride (B) response to consumption of a commercially available high-fat, high-calorie pizza meal. Venous blood samples were collected following an overnight fast and abstention from exercise. Serum samples were analyzed using an automated chemistry analyzer. Subjects consumed an oral spore-based probiotic supplement for 30 days and the experimental meal challenge was completed at baseline and following the 30 day supplementation period. Probiotic responses were compare to placebo. An “a” indicates significantly less than placebo, less than pre-meal, less than and same time point at baseline (P<0.05).

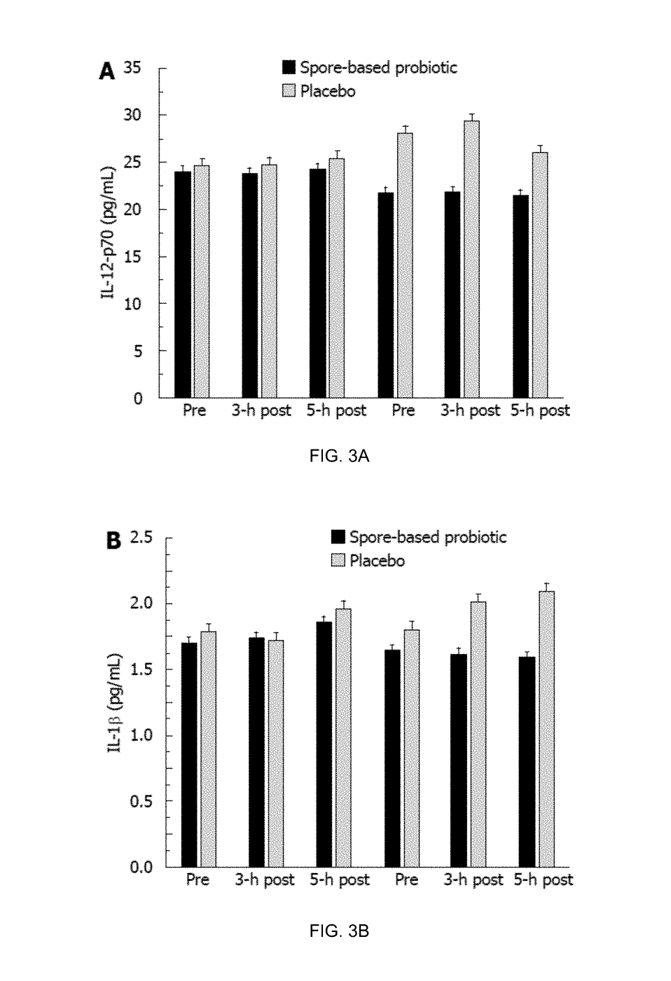

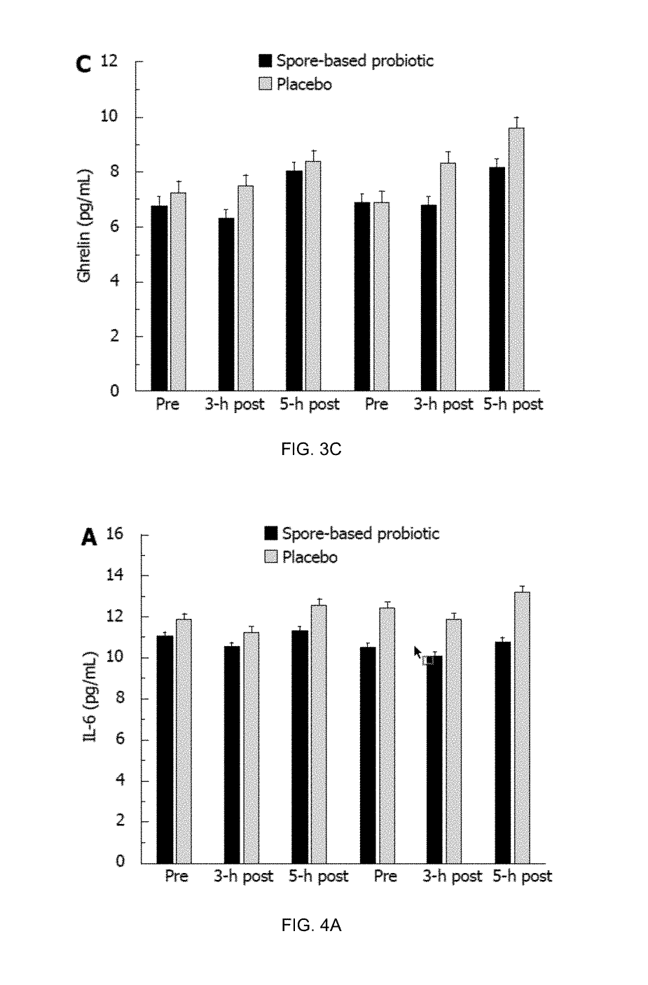

FIGS. 3A-C show serum IL-12p70, IL-13, and post-prandial ghrelin response, respectively, to consumption of a commercially available high-fat, high-calorie pizza meal. Venous blood samples were collected following an overnight fast and abstention from exercise. Serum samples were analyzed using an automated chemistry analyzer. Subjects consumed an oral spore-based probiotic supplement for 30 days and the experimental meal challenge was completed at baseline and following the 30 day supplementation period. Probiotic responses were compare to placebo.

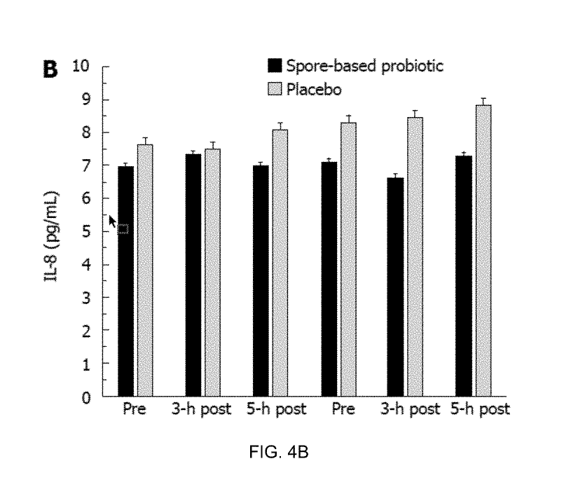

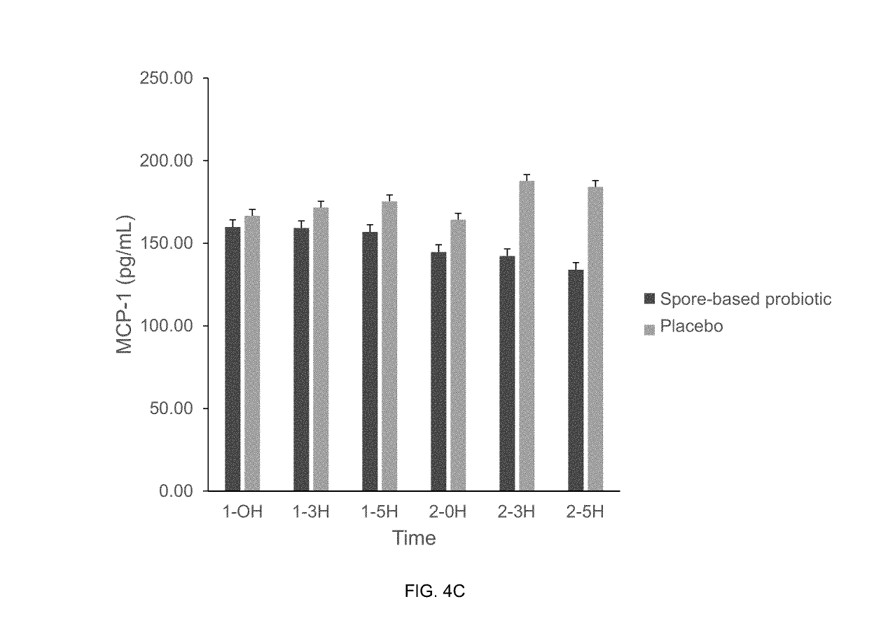

FIGS. 4A-C show serum IL-6, IL-8, and MCP-1 response to consumption of a commercially available high-fat, high-calorie pizza meal. Venous blood samples were collected following an overnight fast and abstention from exercise. Serum samples were analyzed using an automated chemistry analyzer. Subjects consumed an oral spore-based probiotic supplement for 30 days and the experimental meal challenge was completed at baseline and following the 30 day supplementation period. Probiotic responses were compare to placebo. These effects are consistent with the pattern observed for serum endotoxin in that spore-based probiotic intervention was associated with a reduction in a given biomarker at post-supplementation compared to pre-supplementation and placebo.

Results

Endotoxin and Triglycerides: Significant three-way interaction effects were found for both serum endotoxin (P=0.011; FIG. 2A) and triglycerides (P=0.004; FIG. 2B). In each instance, there was no difference between the post-prandial response between the two treatment groups (i.e. placebo vs. spore-based probiotic) at baseline; however, the significant differences were apparent at post-supplementation. Specifically, spore-based probiotic supplementation was associated with a 42% reduction in serum endotoxin at 5 hours post-prandial compared to a 36% increase in placebo at the same time point. Spore-based probiotic supplementation was associated with a 24% reduction in serum triglycerides at 3 hours post-prandial compared to a 5% reduction in placebo at the same time point.

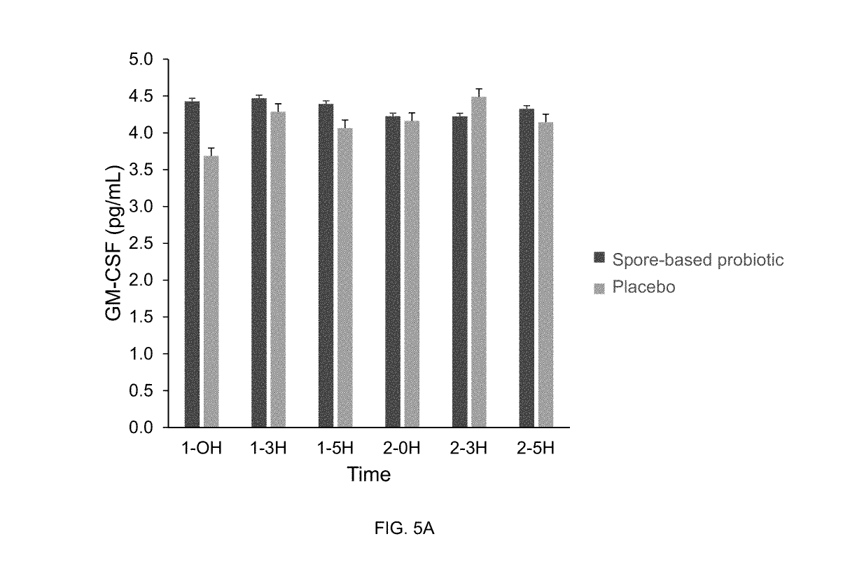

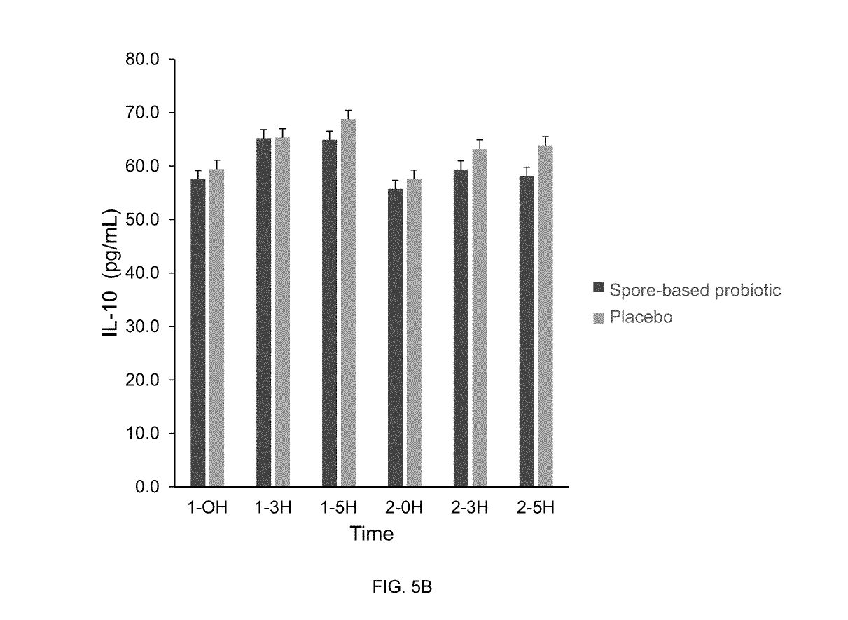

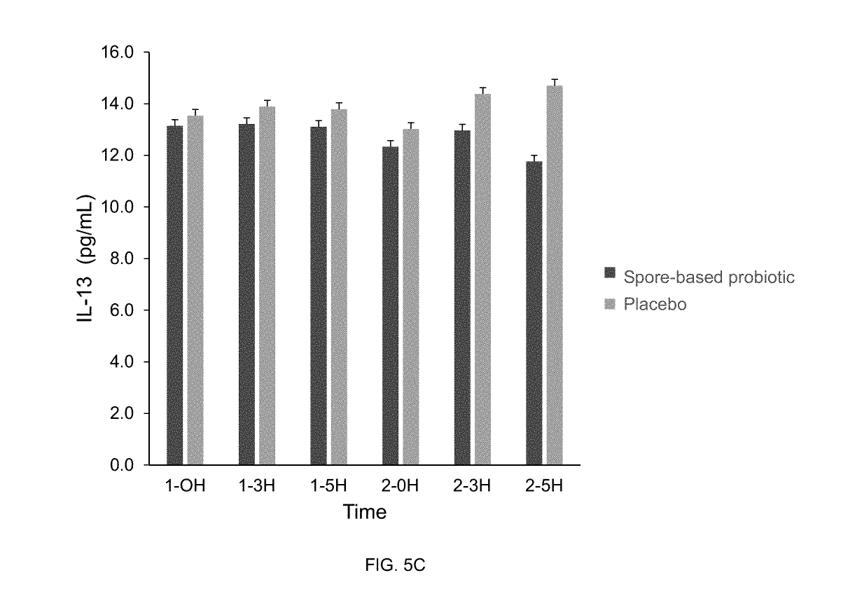

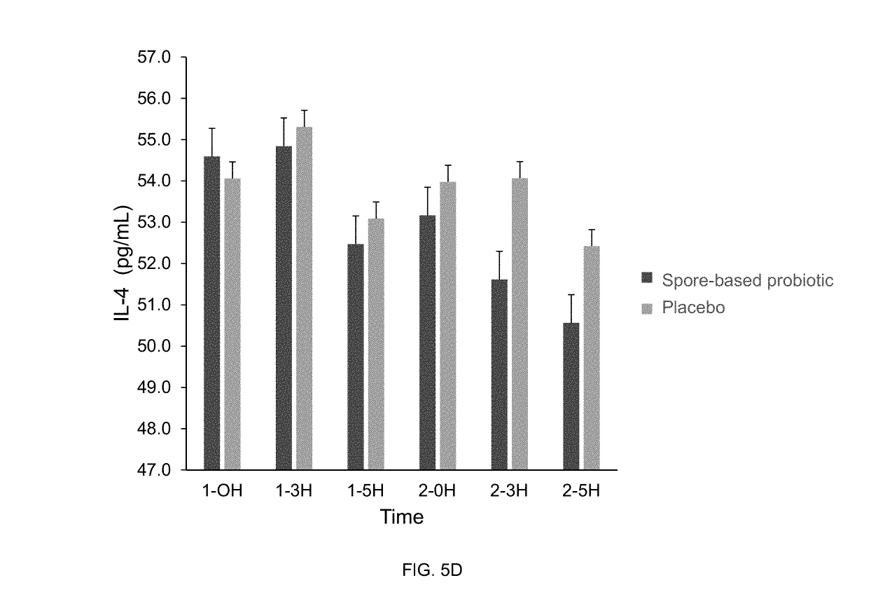

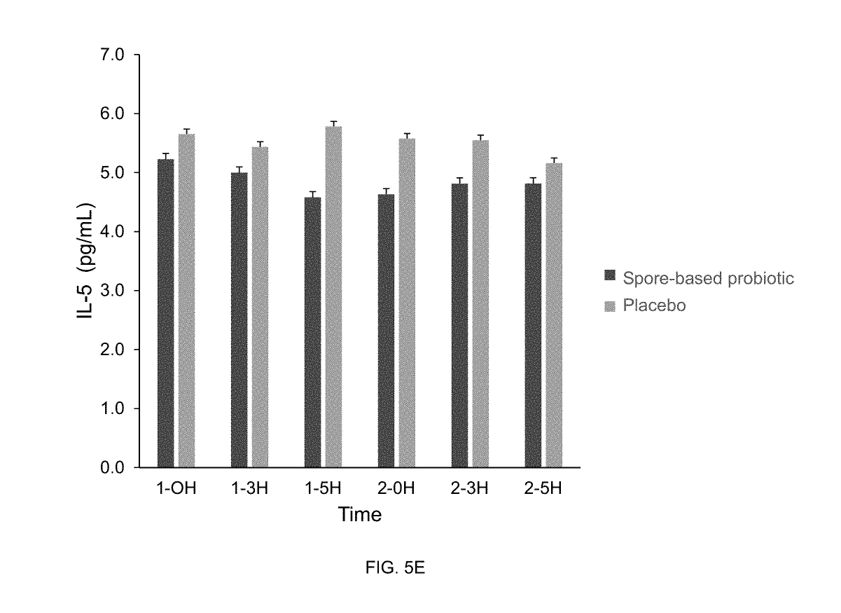

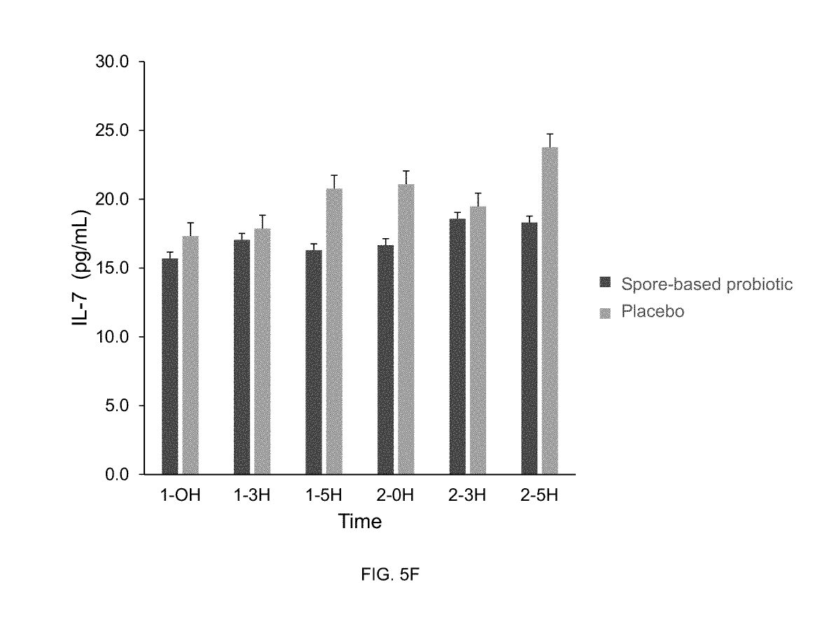

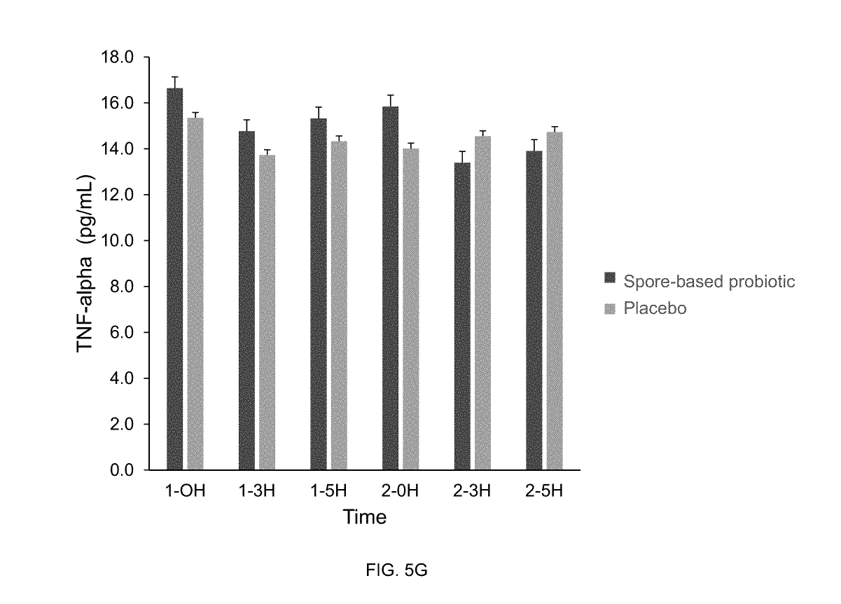

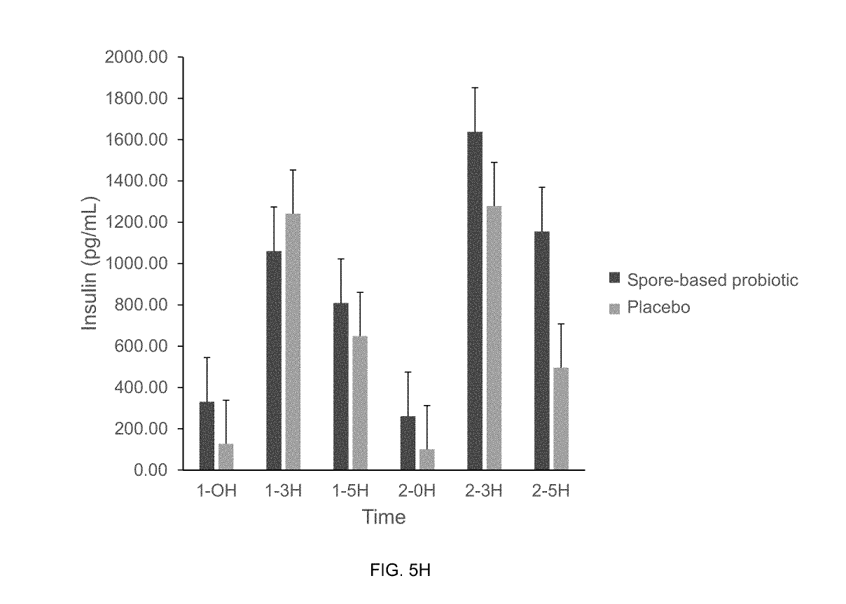

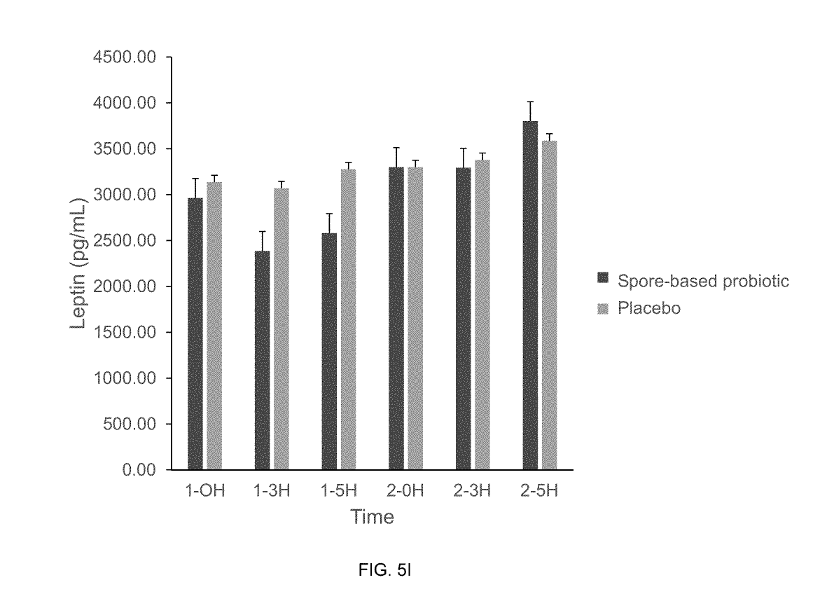

Exploratory Biomarkers: Significant trial×condition interactions for IL-12p70 (P=0.017; FIG. 3A), IL-1β (P=0.020; FIG. 3B), and post-prandial ghrelin (P=0.017; FIG. 3C) were found. Trends for IL-6 (P=0.154; FIG. 4A), IL-8 (P=0.284; FIG. 4B), and MCP-1 (P=0.141; FIG. 4C) were also found. Similar trends of reduction were found for GM-CSF (P=0.159; FIG. 5A), IL-13 (P=0.188; FIG. 5C), IL-4 (P=0.302; FIG. 5D), IL-5 (P=0.973; FIG. 5E), IL-7 (P=0.682; FIG. 5F), TNF-α (P=0.322; FIG. 5G), post-prandial insulin (P=0.128; FIG. 5H) whereas trends of increasing post-prandial levels of leptin (P=0.403; FIG. 5I) and IL-10 (P=0.708; FIG. 5B) were observed. These effects are consistent with the pattern observed for serum endotoxin in that spore-based probiotic intervention is associated with a change in a given biomarker at post-supplementation compared to pre-supplementation and placebo.

The results demonstrate that 30 days of oral supplementation with a viable, spore-based probiotic is associated with a significant reduction in post-prandial endotoxin and triglycerides. Further, several of exploratory biomarkers were either significantly reduced (IL-12p70, IL-1β, and post-prandial ghrelin), trended toward reduction (IL-6, IL-8, and MCP-1, GM-CSF, IL-13, IL-4, IL-5, IL-7, TNF-α, and post-prandial insulin), or trended toward increase (IL-10 and post-prandial leptin) with spore-based probiotic supplementation. Overall, the result was a 42% reduction in metabolic endotoxemia, however, the 30 day of supplementation did not completely prevent metabolic endotoxemia and thus, a longer supplementation period such as for example, 45 days, 60 days, 90 days, etc. is likely to increase the rate of reduction of metabolic endotoxemia or entirely prevent endotoxemia from occurring. Thus, the spore-based probiotic supplement exerted its effect by altering the gut microbial profile, altering intestinal permeability, or a combination of the two effects. These reductions due to spore-based probiotic supplementation are consistent with a transient reduction in chronic disease risk. It should be noted that these reported changes based on the study discussed above were observed while the college-aged subjects continued to lead their habitual life with no directed modification. They continued to be exposed to many of the stressors that are known to negatively affect gut permeability in college-aged individuals (i.e. consumption of microwaved and other processed food, fast foods, soft drinks with excess sugars, including artificial sugars, colorings and flavorings, energy drinks, alcohol consumption, lack of sleep, exam anxiety, etc.).

The placebo subjects presented with an even greater metabolic endotoxemia response following a 30 day period. This may due to a diurnal fluctuation in metabolic endotoxemia responses rather than the experimental treatment. Thus, placebo subjects trended toward increased metabolic endotoxemia, while probiotic intervention reversed that effect.

Previous research has indicated that obese subjects do not have as great of a post-prandial suppression of ghrelin than normal weight subjects. Given the observations of the examples discussed above, it is reasonable to conclude that obesity status may very well affect the gut microbiome. Based on the study parameters described above, without changing body weight, the microbiome of a normal weight individual may be created in an obese individual thus restoring normal post-prandial ghrelin responses.

Given the pro-inflammatory actions of IL-1β, the observed reduction with probiotic supplementation is consistent with reductions in post-prandial systemic inflammation. Reduced post-prandial ghrelin may be indicative of better post-prandial hunger/satiety control with spore-based probiotics. IL-12p70 has a variety of metabolic actions, the chief action in the study of Example 1 is the ability to modulate the release of TNF-α or related inflammatory cytokines following antigenic challenge (11,26). In the case of the study of Example 1, reduced IL-12p70 with spore-based probiotic supplementation may reflect a reduction in systemic inflammatory capacity. In addition to the biomarkers that reached significance, similar numerical trends were found for IL-6, IL-8, and MCP-1, which are all released by adipose tissues and commonly elevated in obese individuals. The biomarkers observed to change in the study of Example 1 following the spore-based probiotic intervention are involved in the accumulation of systemic inflammation. Elevated systemic inflammation has been linked to the pathophysiology of cardiovascular and metabolic diseases, thus even a transient reduction in systemic inflammation biomarkers may be associated with reduced disease risk. The biomarkers measured in the study of Example 1 are most often measured in the context of long-term weight loss (>12 weeks) interventions. In those weight loss models, it can take up to 16-weeks to reduce body weight enough that biomarkers change. Here, similar reductions in inflammatory biomarkers occurred not only in one quarter the time, but also in the absence of weight loss.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A method of treating metabolic endotoxemia comprising:
identifying a subject having metabolic endotoxemia; and
administering an effective amount of a spore-based probiotic.

2. The method of claim 1, wherein the spore-based probiotic comprises spores selected from the group of genus consisting of: Lactobacillus, Bifidobacterium, Lactococcus, Propionibacterium, Bacillus, Enterococcus, Escherichia, Streptococcus, Pediococcus, Saccharomyce.

3. The method of claim 1, wherein the spore-based probiotic comprises spores selected from the group consisting of Bacillus indicus (HU36), Bacillus subtilis (HU58), Bacillus coagulans, Bacillus licheniformis, and Bacillus clausii.

4. The method of claim 1, further comprising reducing at least one of a post-prandial insulin level, a post-prandial ghrelin level, and an MCP-1 level.

5. The method of claim 1, further comprising reducing a level of at least one of GM-CSF, IL-12p70, IL-13, IL-1beta, IL-4, IL-5, IL-6, IL-7, IL-8, and TNF-α.

6. The method of claim 1 further comprising increasing at least one of a post-prandial leptin level and an IL-10 level.

7. The method of claim 1, wherein the spore-based probiotic comprises spores having a survival rate between about 75% and 99% after exposure to gastric acid.

8. The method of claim 1, wherein the spore-based probiotic comprises spores having a survival rate greater than about 90% after exposure to gastric acid.

9. The method of claim 1, wherein the spore-based probiotic is in at least one of a liquid form, a pill form and a food product form.

10. The method of claim 1, wherein the subject experiences at least one of a reduction in triglyceride and a post-prandial reduction of an endotoxin after administration of the effective amount of the spore-based probiotic.

11. The method of claim 10, wherein the endotoxin comprises lipopolysaccharide (LPS).

12. A method of reducing a blood endotoxin level comprising:
identifying a subject having an increased post-prandial level of a blood endotoxin; and
administering an effective amount of a spore-based probiotic.

13. The method of claim 12, wherein the spore-based probiotic comprises spores selected from the group of genus consisting of Lactobacillus, Bifidobacterium, Lactococcus, Propionibacterium, Bacillus, Enterococcus, Escherichia, Streptococcus, Pediococcus, Saccharomyce.

14. The method of claim 12, wherein the spore-based probiotic comprises spores selected from the group consisting of Bacillus indicus (HU36), Bacillus subtilis (HU58), Bacillus coagulans, Bacillus licheniformis, and Bacillus clausii.

15. The method of claim 12, wherein the spore-based probiotic comprises spores having a survival rate between about 75% and 99% after exposure to gastric acid.

16. The method of claim 12, wherein the spore-based probiotic comprises spores having a survival rate greater than about 90% after exposure to gastric acid.

17. The method of claim 12, further comprising reducing at least one of an endotoxin level and a triglyceride level of the subject after administration of the effective amount of the spore-based probiotic.

18. The method of claim 17, wherein the endotoxin comprises lipopolysaccharide (LPS).

19. The method of claim 12, further comprising reducing at least one of a post-prandial insulin level, a post-prandial ghrelin level, and an MCP-1 level after administration of the effective amount of the spore-based probiotic.

20. The method of claim 12, further comprising reducing a level of at least one of GM-CSF, IL-12p70, IL-13, IL-1beta, IL-4, IL-5, IL-6, IL-7, IL-8, and TNF-α after administration of the effective amount of the spore-based probiotic.

21. The method of claim 12, further comprising increasing at least one of a post-prandial leptin level and an IL-10 level after administration of the effective amount of the spore-based probiotic.

22. The method of claim 12, wherein the spore-based probiotic is in at least one of a liquid form, a pill form, and a food product form.
